(19) United States
(12) Patent Application Publication
(10) Pub. No.: US 2023/0347032 A1
(43) Pub. Date: Nov. 2, 2023

(54) MEMBRANE OXYGENATOR

(71) Applicant: Beihang University, Beijing (CN)

(72) Inventors: Zengsheng CHEN, Beijing (CN); Xingji FU, Beijing (CN); Yubo FAN, Beijing (CN); Xiaoyan DENG, Beijing (CN)

(73) Assignee: Beihang University, Beijing (CN)

(21) Appl. No.: 17/993,285

(22) Filed: Nov. 23, 2022

(30) Foreign Application Priority Data
Apr. 28, 2022 (CN) 202210469943.3

(51) Int. Cl. A61M 1/16

(52) U.S. Cl. CPC A61M 1/1698; A61M 1/1621

(57) ABSTRACT

Disclosed is a membrane oxygenator, comprising a housing, and a blood-oxygen exchange chamber arranged in the housing. A liquid inlet side of the housing is provided with at least two blood inlets, and a liquid outlet side of the housing is provided with at least two blood outlets. The liquid inlet side and the liquid outlet side are respectively located at either side of the housing. Projections of blood inlets at the liquid outlet side do not coincide with blood outlets. By designing the shape of the blood-oxygen exchange chamber, blood inlets and blood outlets, and a blood inlet porous baffle of the membrane oxygenator in a mode fits to the shape design of the blood-oxygen exchange chamber and blood inlets, the effects of evenly distributing the blood flow to enable full blood-gas exchange, relieving the blood stasis and the like are achieved, and thus reduce the clinical thrombosis risk.

### CROSS-REFERENCE TO RELATED APPLICATION

This patent application claims the benefit and priority of Chinese Patent Application No. 202210469943.3 filed with the China National Intellectual Property Administration on Apr. 28, 2022, the disclosure of which is incorporated by reference herein in its entirety as part of the present application.

### TECHNICAL FIELD

The present disclosure relates to the technical field of medical devices, and in particular relates to a membrane oxygenator.

### BACKGROUND

Membrane oxygenator, which is a medical device capable of assisting in extracorporeal blood circulation of human body, is commonly used in extracorporeal membrane oxygenation (ECMO) technology, and is an important component of an ECMO system. The membrane oxygenator can contain a certain volume of blood, deoxygenated venous blood drawn from the human body passes through the blood-gas exchange zone formed by a large number of hollow fiber membrane filaments in the membrane oxygenator to be subjected to blood-gas exchange with the oxygen flowing through membrane filaments, and then is converted into oxygenated arterial blood to flow back to the human body again, and therefore the membrane oxygenator takes on the function of human lungs in the extracorporeal circulation.

External structure design of the membrane oxygenator has an important impact on its internal blood flow field, such that a plurality of membrane oxygenators with different configurations and different performance are available in market, The purpose of improving the safety and functionality of the membrane oxygenators is finally achieved by changing the external structure of membrane oxygenators, and thus affecting the blood flow features in the membrane oxygenators, which is an important technology for the research and development of the membrane oxygenators at present.

There are many different types of membrane oxygenators with different structures. For a membrane oxygenator with better clinical performance at present, namely QUADROX series oxygenator developed by Maquet Group, as shown in FIG. 1, its blood-gas exchange zone is modeled into a rectangular cuboid shape, and the blood inlet and the blood outlet mostly employ a “one-to-one” design, namely, a single and coaxial inlet and outlet design.

Clinical treatment often requires membrane oxygenators to adopt the single and coaxial inlet and outlet design for ease of connection. However, due to the fact that the membrane oxygenators are large in size and small in size of the blood inlet and the blood outlet, the use of such design may lead to uneven distribution of the blood flow rate, and all blood flow rate only flows in and out in a certain region of a blood-gas exchange zone in a centralized mode, while the rest of regions far away from the blood inlet and the blood outlet lack the direct inflow and outflow of the blood flow rate. The blood, if desired to flow through these regions away from the blood inlet and the blood outlet, requires a longer path within the membrane oxygenators, which results in blood stasis. The experimental results also showed that, the blood tends to flow more smoothly in the regions closer to the blood inlet and the blood outlet, while some blood is retained in the regions far away from the blood inlet and the blood outlet.

In the membrane oxygenators, two flowing states may lead to corresponding problems: fast outflow of the blood may lead to insufficient blood-gas exchange, while blood stasis leads to thrombosis, which further blocks flow of the blood and reduces the blood-gas exchange performance. Existing research results have also shown that thrombosis is often severe in sharp corner regions of the rectangular cuboid-shaped blood-gas exchange zone, as well as in the regions far away from the blood inlet and the blood outlet, of the QUADROX series membrane oxygenators. Therefore, how to solve insufficient blood-gas exchange and thrombosis caused by uneven distribution of blood flow rate is an important problem needing to be considered when designing the membrane oxygenators.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

An objective of the present disclosure is to provide a membrane oxygenator. By designing the housing and porous baffles of the membrane oxygenator, the insufficient blood-gas exchange and thrombosis caused by uneven distribution of blood flow volume are solved.

To solve the problems above, the present disclosure provides a membrane oxygenator. The membrane oxygenator comprises a housing and a blood-oxygen exchange chamber located in the housing.

A liquid inlet side of the housing is provided with at least two blood inlets, a liquid outlet side of the housing is provided with at least two blood outlets, and the liquid inlet side and the liquid outlet side are located at either side of the housing.

Projections of the blood inlets at the liquid outlet side do not coincide with positions of the blood outlets, thus enabling the blood flow flowing out from the membrane oxygenator to be even.

Preferably, the blood-oxygen exchange chamber is in a shape of cylinder or rectangular cuboid.

Preferably, the blood inlets are distributed in a first circumference, and the blood outlets are distributed in a second circumference.

The center of the first circumference and the center of the second circumference are both located on an axis of the blood-oxygen exchange chamber.

The diameter of the first circumference is not greater than that of the second circumference.

Preferably, the blood inlets and the blood outlets are evenly distributed at equal angles along the first circumference and the second circumference, respectively.

Preferably, the liquid outlet side of the housing is further provided with a blood central outlet, and the blood central outlet is located at a center of the circle of the second circumference.

Preferably, the liquid inlet side of the housing is provided with a main blood inlet, the liquid outlet side of the housing is provided with a main blood outlet, and the main blood inlet is coaxial with the main blood outlet.

The main blood inlet is connected to the blood inlets.

The main blood outlet is connected to the blood outlets.

Preferably, branch ends, connected to the blood inlets, of the main blood inlet each are provided with a vertical pipeline, the vertical pipeline is perpendicular to an side surface of the liquid inlet side, and is configured for controlling a radial sub-speed of the blood when entering an inlet porous baffle.

Preferably, the blood-oxygen exchange chamber comprises a hollow fiber membrane tow and an inlet porous baffle for pressing the hollow fiber membrane tow.

The inlet porous baffle comprises blood flow inlet through-hole zones, blood flow convergence through-hole zones, and other through-hole zones.

The blood flow inlet through-hole zones are located at the blood inlets and configured for changing flow directions of the blood at the blood inlets.

The blood flow convergence through-hole zones are located at convergence of a plurality of blood flows and configured for dredging the blood.

The other through-hole zones are configured for controlling the blood flow rate.

Preferably, the blood flow inlet through-hole zones each are provided with a central through hole, and the area of the central through hole is smaller than that of the blood inlet.

The diameter of the through hole at each blood flow convergence through-hole zone is greater than that of the central through hole.

Preferably, the blood-oxygen exchange chamber further comprises an outlet porous baffle in which a plurality of through holes having a same diameter are evenly provided.

The above technical solution of the present disclosure has the following beneficial technical effects:

By designing the shape of the blood-oxygen exchange chamber, the blood inlets and the blood outlets, and the inlet porous baffle of the membrane oxygenator in a mode fits to the design of the shape of the blood-oxygen exchange chamber and the blood inlets, the effects of evenly distributing the blood flow rate to enable full blood-gas exchange, relieving the blood stasis and the like are achieved, and thus complications such as thrombosis and the like can be prevented clinically.

### BRIEF DESCRIPTION OF THE DRAWINGS

The foregoing summary, as well as the following detailed description of presently preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1 is a structure diagram of a QUADROX series membrane oxygenator in the prior art;

FIGS. 2A-2B is a structure diagram of a housing of a membrane oxygenator in accordance with the present disclosure;

FIG. 3 schematically shows the distribution of blood inlet positions;

FIG. 4 schematically shows the distribution of blood outlet positions;

FIG. 5 is a cross-sectional view of an inlet porous baffle in accordance with one embodiment of the present disclosure;

FIG. 6 is a cross-sectional view of an outlet porous baffle in accordance with one embodiment of the present disclosure;

FIG. 7 is a distribution diagram of accumulated residence time at an inlet buffer zone of a blood-oxygen exchange chamber; and

FIG. 8 is a distribution diagram of accumulated residence time at an inlet buffer zone of a blood-oxygen exchange chamber improved in accordance with one embodiment of the present disclosure.

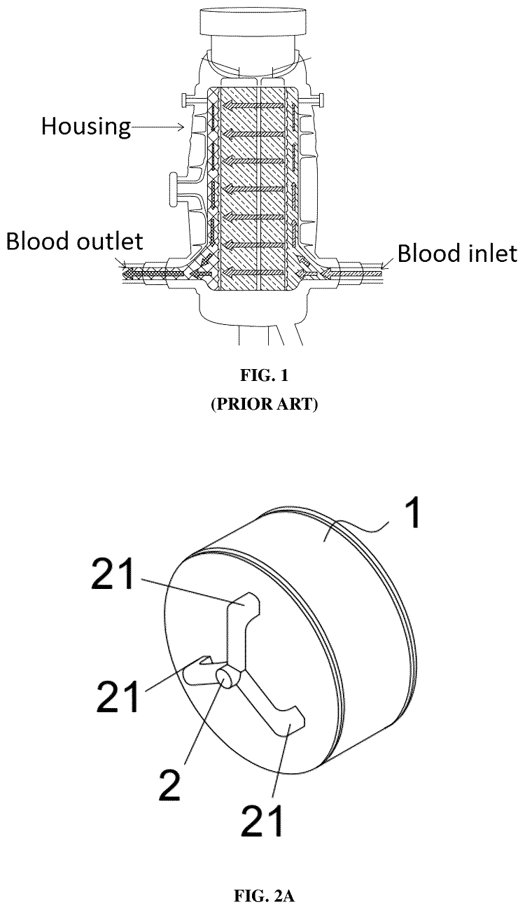

To facilitate an understanding of the invention, identical reference numerals have been used, when appropriate, to designate the same or similar elements that are common to the figures. Further, unless stated otherwise, the features shown in the figures are not drawn to scale and are shown for illustrative purposes only.

In the drawings:
1—Housing;
2—Main blood inlet; 21—blood inlet
3—Main blood outlet; 31—blood outlet; 32—blood center outlet;
4—Inlet porous baffle; 41—blood flow inlet through-hole zone; 411—central through hole; 42—blood flow convergence through-hole zone; 43—other through-hole zones.

### DETAILED DESCRIPTION OF THE EMBODIMENTS

To make the objectives, technical solution and advantages of the present disclosure more clearly, the following further describes the present disclosure in detail in conjunction with specific embodiments and with reference to the accompanying drawings. It should be understood that these descriptions are illustrative and not intended to limit the scope of the present disclosure. In addition, in the following, well-known structures and technologies are not described to avoid obscuring the present disclosure unnecessarily.

Certain terminology is used in the following description for convenience only and is not limiting. The article “a” is intended to include one or more items, and where only one item is intended the term “one” or similar language is used. Additionally, to assist in the description of the present invention, words such as top, bottom, side, upper, lower, front, rear, inner, outer, right and left are used to describe the accompanying figures. The terminology includes the words above specifically mentioned, derivatives thereof, and words of similar import.

In the description of the present disclosure, it should be noted that the terms “first”, “second” and “third” are used for descriptive purposes only and are not to be construed as indicating or implying relative importance. The described embodiments are only part rather than all of the embodiments of the present disclosure. On the basis of the embodiments of the present disclosure, all other embodiments acquired by those of ordinary skill in the art without making inventive efforts fall within the scope of the present disclosure.

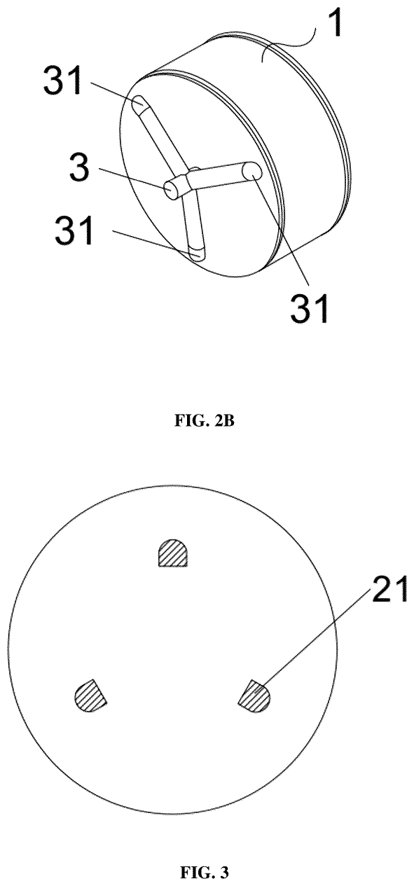

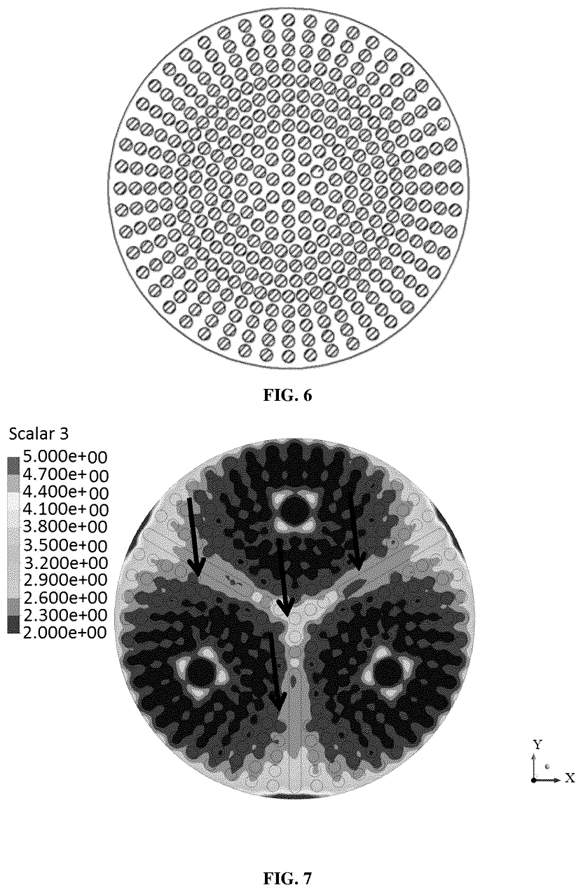

With reference to FIGS. 2A-2B, a membrane oxygenator of the present disclosure is described in detail in conjunction with structure diagrams of various parts in FIG. 3 to FIG. 6.

Two opposite side surfaces of a housing 1 of the membrane oxygenator are a liquid inlet side and a liquid outlet side. The liquid inlet side is provided with at least two blood inlets 21 having the same size, and the liquid outlet side is provided with at least two blood outlets 31 having the same size. The number of the blood inlets 21 is the same as the number of the blood outlets 31, and the spatial positions of the blood inlets and the blood outlets are staggered, that is, projections of the blood inlets 21 at the liquid outlet side do not coincide with the blood outlets 31, or projections of the blood outlets 31 at the liquid inlet side do not coincide with the blood inlets 21.

Such design allows the blood to flow in the blood-oxygen exchange chamber and to flow out of the blood-oxygen exchange chamber from different regions, so as to achieve the effect of reasonably distributing the blood flow rate at all positions in the membrane oxygenator. Meanwhile, by means of the design that each pair of blood inlet 21 and blood outlet 31 is reasonably staggered by a certain distance in a circumferential direction, the path lengths of the blood flows which flow out from the membrane oxygenator may be averaged, such that the path lengths of the blood flows in the membrane oxygenator are similar as much as possible, thus relieving the blood stasis.

The blood-oxygen exchange chamber is located in the housing 1, and comprises an inlet porous baffle 4, a hollow fiber membrane tow and an outlet porous baffle which are adaptive to the internal size of the housing 1. The inlet porous baffle 4 is used for pressing the hollow fiber membrane tow and forming a cavity of the blood-oxygen exchange chamber with an inner wall of the housing 1 and the outlet porous baffle, such that the blood entering from the blood inlet 21 flows in the blood-oxygen exchange chamber through the inlet porous baffle 4 and then flows out of the membrane oxygenator through the outlet porous baffle and the blood outlets 31 to complete the blood-oxygen exchange.

By designing the through holes of the inlet porous baffle, the secondary distribution of the blood flows is achieved, and the blood stasis in the blood-oxygen exchange chamber is relieved, and thus the flow features and functionality of the membrane oxygenator are improved, and thrombosis is prevented.

The blood-oxygen exchange chamber may be in a shape of rectangular cuboid or cylinder.

In a preferred embodiment of the present disclosure, the blood-oxygen exchange chamber is designed as a cylinder, and the corresponding housing is also a cylinder. Compared with the design of the rectangular cuboid-shaped chamber, the cylinder may effectively relieve the blood stasis caused by the sharp corner regions of the rectangular cuboid.

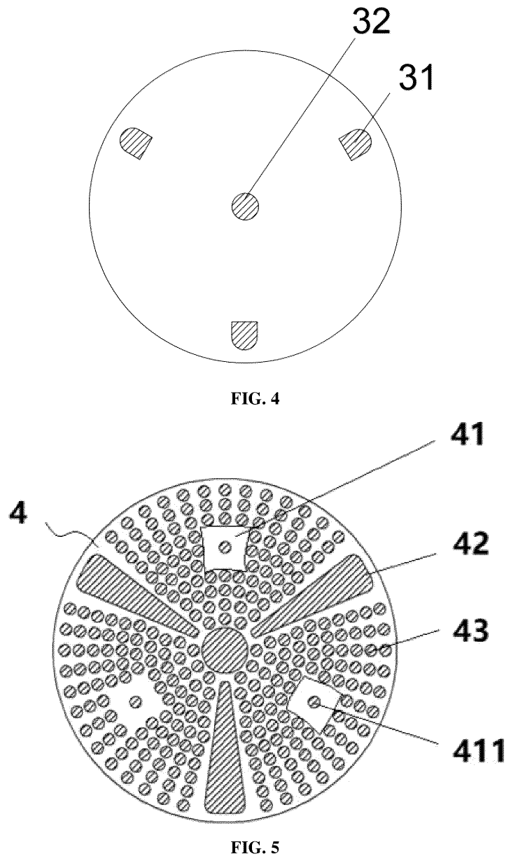

Furthermore, the liquid inlet side and the liquid outlet side of the housing 1 are respectively provided with three blood inlets 21 and three blood outlets 31 which are equal in size and are cylindrical through holes, referring to FIG. 3 and FIG. 4.

The center of a first circumference where the three blood inlets 21 on the liquid inlet side are located and the center of a second circumference where the three blood outlets 31 on the liquid outlet side are located are both located on an axis of the blood-oxygen exchange chamber, and the blood inlets 21 and the blood outlets 31 are evenly distributed at equal angles along the first circumference and the second circumference, respectively.

To ensure that blood can fully flow through the blood-oxygen exchange chamber and the path lengths of the blood flows in the membrane oxygenator are similar, the radius of the first circumference is set to be smaller than the radius of the second circumference, and the centers of each pair of blood inlet 21 and blood outlet 31 are not coaxial and are mutually staggered. Preferably, the three blood inlets 21 are arranged at a position ⅓ radius away from the edge of the cylinder.

Furthermore, in order to average the path lengths of the blood flows, a blood center outlet 32 is provided in the center of the housing 1 to allow the blood in a region, where no blood directly flows in, to flow out in time, thereby relieving the blood stasis at regions away from the blood inlet.

Furthermore, for ease of connection in clinical use, a design of single and coaxial main inlet and main outlet is adopted, that is, a main blood inlet 2 and a main blood outlet 3 are designed at the liquid inlet side and the liquid outlet side of the housing 1, respectively. By means of the one-to-many pipelines, the main blood inlet 2 is connected to the blood inlets 21, and the main blood outlet 3 is connected to the blood outlets 31 and the blood center outlet 32.

Meanwhile, branch pipelines connected to the three blood inlets each are provided with a pipeline vertical to the blood inlets with a height of 10 mm, the pipeline vertical to the blood inlets is perpendicular to the side surface of the liquid inlet side, and the other end of the pipeline vertical to the blood inlets is connected to the main blood inlet 2. By means of the design of the pipeline vertical to the blood inlets, the blood does not generate radial sub-speed and still flows into the membrane oxygenator in an axial direction of the cylindrical housing 1.

To enhance the effect of distributing the blood flow rate, the inlet porous baffle 4 for blood is further designed. On the one hand, the inlet porous baffle is configured to press the flexible hollow fiber membrane tow to reduce the deformation of the flexible hollow fiber membrane tow caused by the impact of the blood flow. On the other hand, by designing the position and size of the through holes on the inlet porous baffle, the secondary distribution of the flood flow rate at the blood inlets is achieved, and the effect of controlling flow of the blood is achieved.

In a second embodiment of the present disclosure, analysis software Ansys is used for performing numerical simulation analysis on flow fields when the blood reaches the blood-oxygen exchange chamber through the blood inlets 21, and the inlet porous baffle 4 is divided into blood flow inlet through-hole zones 41, blood flow convergence through-hole zones 42 and other through-hole zones 43 based on the simulation result.

The positions of the blood flow inlet through-hole zones 41 correspond to the positions of the blood inlets 21, the number of the through holes at the blood flow inlet through-hole zones 41 is reduced, only one through hole is reserved, and the other positions are replaced with a baffle. By means of the blocking effect of the baffle on the blood, flow direction of part of the blood is forced to change from an axial direction to a radial direction and then the part of the blood flows to a region away from the blood inlets, thereby achieving the effect of secondary distribution of the blood flow rate.

Blood flow convergence through-hole zones 42 are located in regions where a plurality of blood flows converge, the blood stasis is prone to occurring at these regions. Therefore, in order to improve the blood stasis in these regions, through holes in these regions are designed to be larger to dredge flow of the blood, thereby improving the blood stasis.

Other regions on the inlet porous baffle 4 are the other through-hole zones 43. The size of through holes may be designed based on the obtained simulation data, for example, the diameter of the through holes at a region away from the blood inlets is increased to facilitate the flow of blood.

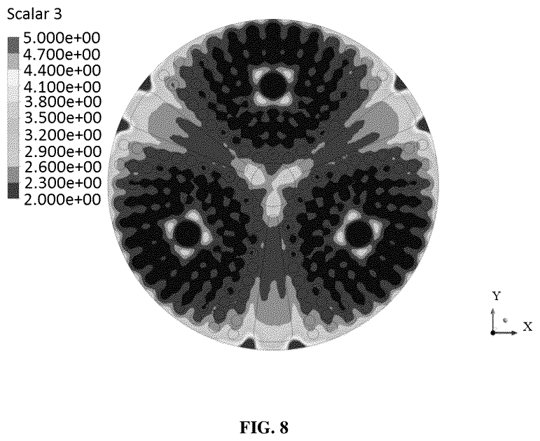

FIGS. 7-8 are diagrams of simulation obtained using software, accumulated residence time is chosen as a parameter for assessing blood stasis, and such parameter has a physical meaning referring to the time required for the blood to flow from the blood inlets to a particular position.

At first, the flow of the blood in the membrane oxygenator is simulated to obtain the distribution of accumulated residence time at an inlet buffer zone of the blood-oxygen exchange chamber as shown in FIG. 7, it can be known from the physical meaning of the accumulated residence time that high value regions indicated by arrows denote more severe blood stasis, so it is contemplated that the processing of enlarging the through holes of the inlet porous baffle is employed in these regions to dredge stagnated blood.

The distribution of the accumulated residence time on the same scalar with the membrane oxygenator as shown in FIG. 7 after the enlargement of through holes is shown in FIG. 8. It is easy to see that the reasonable enlargement and arrangement of the through holes has improved the effect in relieving blood stasis.

In a third embodiment of the present disclosure, under the dual action of the blood inlets 21 and the inlet porous baffle 4, the blood flow rate has been reasonably distributed. Meanwhile, the hollow fiber membrane tow may provide a large flow resistance, thus leading to low flow rate of the blood, and complex flowing state does not occurs, and therefore at the blood outlets, it is possible to make only conventional design for the outlet porous baffle which is provided with evenly distributed through holes.

The present disclosure is intended to protect a membrane oxygenator. One the one hand, the blood inlets and the blood outlets of the membrane oxygenator are designed based on three principles of “a plurality of ports, distribution and staggering”, among which, “the plurality of ports” and the “distribution” specifically refer to that a plurality of blood inlets and blood outlets are provided and distributed in different regions of the blood-gas exchange zone. Such design allows the blood to flow in from, or out of, the different regions respectively, thus achieving the effect of reasonably distributing the blood flow rate at all positions of the membrane oxygenator, and the “staggering” specifically refers to that the design of the blood inlets and the blood outlets on the housing does not employs a “many-to-many” inlet-outlet coaxial design similar to “one-to-one” inlet-outlet coaxial design, but the design that each pair of inlets and outlets is staggered at a reasonable distance, such design allows the path lengths of the blood flows in the membrane oxygenator to be as similar as possible, thereby relieving blood stasis.

On the other hand, in several regions where blood hedging and blood stasis are predicted to occur, the method of changing the shapes of the through holes and enlarging the diameters of the through holes is used to dredge the blood in the region of blood stasis in the premise of not seriously affecting the action of pressing the hollow fiber membrane tow, such that the blood can flow out easier, and the condition of blood stasis is relieved. Therefore, the thrombosis, the common complication of the oxygenator, is prevented while the fluidity and functionality of the membrane oxygenator are improved.

It should be understood that the above detailed description of the present disclosure is intended only to illustrate or explain the principles of the present disclosure rather than constituting the limitation of the present disclosure. Accordingly, any modifications, equivalents, improvements, and the like made without departing from the spirit and scope of the present disclosure should be included within the scope of the present disclosure. In addition, the appended claims of the present disclosure are intended to cover all the changes and modifications falling within the scope and boundary, or the equivalents thereof, of the appended claims.

What is claimed is:

1. A membrane oxygenator, comprising a housing (1) and a blood-oxygen exchange chamber located in the housing (1);
a liquid inlet side of the housing (1) is provided with at least two blood inlets (21), a liquid outlet side of the housing (1) is provided with at least two blood outlets (31), and the liquid inlet side and the liquid outlet side are located at either side of the housing (1) respectively;
projections of the blood inlets (21) at the liquid outlet side do not coincide with the blood outlets (31), thus enabling a blood flow flowing out from the membrane oxygenator to be even.

2. The membrane oxygenator according to claim 1, wherein the blood-oxygen exchange chamber is in a shape of cylinder or rectangular cuboid.

3. The membrane oxygenator according to claim 1, wherein the blood inlets (21) are distributed in a first circumference, and the blood outlets (31) are distributed in a second circumference;
the center of the first circumference and the center of the second circumference are both located on an axis of the blood-oxygen exchange chamber; and
the diameter of the first circumference is not greater than that of the second circumference.

4. The membrane oxygenator according to claim 3, wherein the blood inlets (21) and the blood outlets (31) are evenly distributed at equal angles along the first circumference and the second circumference, respectively.

5. The membrane oxygenator according to claim 3, wherein the liquid outlet side of the housing (1) is further provided with a blood central outlet (32), and the blood central outlet (32) is located at a center of the circle of the second circumference.

6. The membrane oxygenator according to claim 4, wherein the liquid inlet side of the housing (1) is provided with a main blood inlet (2), the liquid outlet side of the housing (1) is provided with a main blood outlet (3), and the main blood inlet (2) is coaxial with the main blood outlet (3);
the main blood inlet (2) is connected to the blood inlets (21); and
the main blood outlet (3) is connected to the blood outlets (31).

7. The membrane oxygenator according to claim 6, wherein branch ends, connected to the blood inlets (21), of the main blood inlet (2) each are provided with a vertical pipeline, the vertical pipeline is perpendicular to an side surface of the liquid inlet side, and is configured for controlling a radial sub-speed of the blood when entering an inlet porous baffle (4).

8. The membrane oxygenator according to claim 1, wherein the blood-oxygen exchange chamber comprises a hollow fiber membrane tow and an inlet porous baffle (4) for pressing the hollow fiber membrane tow;
the inlet porous baffle (4) comprises blood flow inlet through-hole zones(41), blood flow convergence through-hole zones (42), and other through-hole zones (43);
the blood flow inlet through-hole zones are located at the blood inlets (21) and configured for changing flow directions of the blood at the blood inlets (21);
the blood flow convergence through-hole zones (42) are located at convergence of a plurality of blood flows and configured for dredging the blood; and
the other through-hole zones (43) are configured for controlling blood flow rate.

9. The membrane oxygenator according to claim 8, wherein the blood flow inlet through-hole zones (41) each are provided with a central through hole (411), and the area of the central through hole (411) is smaller than that of the blood inlet (21);
the diameter of the through hole at each blood flow convergence through-hole zone (42) is greater than that of the central through hole (411).

10. The membrane oxygenator according to claim 8, wherein the blood-oxygen exchange chamber further comprises an outlet porous baffle in which a plurality of through holes having a same diameter are evenly provided.

11. The membrane oxygenator according to claim 2, wherein the blood inlets (21) are distributed in a first circumference, and the blood outlets (31) are distributed in a second circumference;
the center of the first circumference and the center of the second circumference are both located on an axis of the blood-oxygen exchange chamber; and
the diameter of the first circumference is not greater than that of the second circumference.

12. The membrane oxygenator according to claim 11, wherein the blood inlets (21) and the blood outlets (31) are evenly distributed at equal angles along the first circumference and the second circumference, respectively.

13. The membrane oxygenator according to claim 11, wherein the liquid outlet side of the housing (1) is further provided with a blood central outlet (32), and the blood central outlet (32) is located at a center of the circle of the second circumference.

14. The membrane oxygenator according to claim 12, wherein the liquid inlet side of the housing (1) is provided with a main blood inlet (2), the liquid outlet side of the housing (1) is provided with a main blood outlet (3), and the main blood inlet (2) is coaxial with the main blood outlet (3);
the main blood inlet (2) is connected to the blood inlets (21); and
the main blood outlet (3) is connected to the blood outlets (31).

15. The membrane oxygenator according to claim 14, wherein branch ends, connected to the blood inlets (21), of the main blood inlet (2) each are provided with a vertical pipeline, the vertical pipeline is perpendicular to an side surface of the liquid inlet side, and is configured for controlling a radial sub-speed of the blood when entering an inlet porous baffle (4).

16. The membrane oxygenator according to claim 2, wherein the blood-oxygen exchange chamber comprises a hollow fiber membrane tow and an inlet porous baffle (4) for pressing the hollow fiber membrane tow;
the inlet porous baffle (4) comprises blood flow inlet through-hole zones(41), blood flow convergence through-hole zones (42), and other through-hole zones (43);
the blood flow inlet through-hole zones are located at the blood inlets (21) and configured for changing flow directions of the blood at the blood inlets (21);
the blood flow convergence through-hole zones (42) are located at convergence of a plurality of blood flows and configured for dredging the blood; and
the other through-hole zones (43) are configured for controlling blood flow rate.

17. The membrane oxygenator according to claim 16, wherein the blood flow inlet through-hole zones (41) each are provided with a central through hole (411), and the area of the central through hole (411) is smaller than that of the blood inlet (21);

18. The membrane oxygenator according to claim 16, wherein the blood-oxygen exchange chamber further comprises an outlet porous baffle in which a plurality of through holes having a same diameter are evenly provided.
